(19) United States
(12) Patent Application Publication
(10) Pub. No.: US 2002/0025507 A1
(43) Pub. Date: Feb. 28, 2002

(54) POLYMERS AS A SUPPORT FOR COMBINATORIAL SYNTHESIS

(76) Inventor: YONG PAN, FAIRFIELD, OH (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(21) Appl. No.: 09/230,076

(22) PCT Filed: Jul. 18, 1997
(86) PCT No.: PCT/US97/12694

(51) Int. Cl. G01N 33/53; C12Q 1/00; C07H 21/02; C07H 21/04; C12P 21/04

(52) U.S. Cl. 435/4; 530/331; 536/25.3

(57) ABSTRACT

“Threedimensional” polymers of very small size can be used in the context of synthesis. These polymers provide simple isolation routes by avoiding the need for workup following the reactions, preworkup characterization, high yield, high capacity and reusability. Of equal importance, these polymers provide a more efficient route to combinatorial synthesis of small molecules. This provides greatly enhanced efficiency for making and screening molecules for physical/biological properties.

### FIELD OF THE INVENTION

This invention relates to the fields of polymers as supports for synthesis, to their methods of making, and to their uses.

### BACKGROUND

Traditionally the organic chemist used suspensions or solutions of reactants to produce the compounds of interest. The approach has the advantages of allowing some analysis of the reaction's progression without “working up” the reaction or purifying the product. In addition, in the context of medicinal chemistry some screening of compounds can be used in evaluating these compounds even though they are not completely purified.

However, the method has certain drawbacks, such as the rigorous control of reaction conditions, extensive purification of product (work up) and the like. These methods tended to be labor intensive and used a large amount of solvents and other chemicals. Often unreacted starting materials and catalysts are not recoverable, and one compound is made at a time.

In the context of medicinal chemistry, or specialty chemistry, production of one compound at a time, and its testing, with attendant serial analysis of the result and design of the next compound is time consuming.

Natural products screening in the pharmaceutical field has shown the need to rapidly evaluate large numbers of molecules in an effort to provide the next generation of therapies. In addition, in the specialty chemical fields, it has been found that screening of large numbers of molecules in parallel, for beneficial properties, is of course more useful than the serial approach. It would be advantageous to make several different compounds at once.

When organic reactions are scaled up, there is a desire to decrease the labor and materials involved. As a result many processes have resorted to solid phase catalysis or solid phase support reactions to achieve greater efficiency. For example, catalytic solid support synthesis has been used in the art of cracking hydrocarbons and for other catalytic reactions.

Recently solid supports for synthesis of molecules has become important in controlling polymer synthesis. By anchoring one end of a living polymer to a solid support, it has been possible to propagate the polymer in only one direction, and in some instances provide a high yield of polymers where the polymer chain is diverse, specified, and well characterized. In these cases, polymer supports, such as functionalized polystyrene beads have been used for such different applications. Based on the type of functionality on the bead there can be any number of moieties attached, reacted, and then removed.

A recent development has been the use of polymer bead supports for the automated synthesis of DNA and polypeptides. In such an application the polymeric beads are packed into a “chromatography column” like shell, and then reagents are added stepwise to propagate the DNA or polypeptide molecules. Finally, an acid, oxidizer or base is added to cleave the polymer chain from the support.

This approach provides a high yielding and convenient alternative to solution phase synthesis. An advantage of the method is that it avoids the need to purify the material being made at each individual step in the synthesis. As a result there has been a desire to use this technology in other applications as well.

In addition, this solid support method has been used to provide multiple polymers for use or screening. For example, combinatorial (or “random”) DNA oligomers have been used in “random PCR”, sequencing the human genome.

However, this approach is used primarily where there is a polymer with only two reactive sites on each monomer (one of which is anchored to the support or living polymer), and a stepwise chemistry is involved. For example, the DNA synthesizer techniques have not been used commercially in synthesizing RNA because of the additional functional site, the 2′ ribose hydroxyl, which provides for undesired side reactions.

In addition, it has been found that “columns” of functionalized beads have a finite lifetime, and replacement costs are high. Thus this method of making molecules has only been employed in limited areas where it is cost prohibitive to make the molecules in another fashion. More importantly, the solid phase synthesis method usually requires expensive equipment, thus there is a desire to decrease the cost associated with the method.

Finally, there has been a finite capacity for solid phase methods used to date. Unlike the solution method of making a compound (where the entire reaction vessel may be filled with reactants if the reaction is run neat), solid phase methods use a large part of the reaction vessel in inert solid phase bulk, thus decreasing the capacity of the reaction vessel. Many of these “support based” processes provide nanograms of material at great cost. Hence there is a desire to increase capacity of the reaction vessel. One way to this is to increase a bead surface area by decreasing the size of the bead. However, this tact introduces problems of flow, pressure buildup, and other adverse phenomena which are common in support based column reactions or liquid chromatography.

In either of solid support or liquid phase synthesis, it has been difficult to determine the reaction products and yield without attendant work up of each reaction and proper isolation of the desired product. It would be convenient to determine the reaction products prior to intensive work up and isolation.

### “THREE-DIMENSIONAL POLYMERS”

The art contains reports of over 30 families of three-dimensional polymers. For example, many were summarized in Topics in Current Chemistry, Vol. 165, 1993, p193 by D. A. Tomalia and H. D. Durst. It would be desirable to make use of the three-dimensional polymer concept in “support based” synthesis.

### OBJECTS OF THE INVENTION

It is an object of the invention to combine the advantages of liquid phase or continuous phase combinatorial synthesis with the advantages of solid phase combinatorial synthesis.

It is an object of the invention to provide a support for synthesis that provides a high yielding and convenient alternative which is cost effective to solution phase synthesis which is less expensive than solid support synthesis.

It is an object of the invention to provide a support for synthesis that avoids the need for extensive work up in the context of making and isolating molecules before characterization. For example, it is an object of the invention to provide a “support” that allows the skilled artisan to perform spectroscopy before isolation including NMR, IR, and UV by conventional means.

It is an object of the invention to provide a soluble “support” for synthesis that is reusable and does not require elaborate equipment to achieve solid phase results.

It is an object of the invention to provide a “support” for synthesis that provides a higher capacity compared to solid phase synthesis.

### SUMMARY OF THE INVENTION

It has been found that “three-dimensional” polymers of very small size can be used in the context of synthesis. These polymers provide:

(1) simple isolation routes by avoiding the need for workup following the reactions;

(2) pre-workup characterization;

(3) high yield

(4) high capacity; and

(5) reusability.

Of equal importance, these polymers provide a more efficient route to combinatorial synthesis of small molecules. This provides greatly enhanced efficiency for making and screening molecules for physical/biological properties.

### DETAILED DESCRIPTION

Since many analytical methods and screening methods are solvent or solution based, the invention advantageously circumvents the need to purify compounds made using the method of the invention.

The invention uses a “three-dimensional polymer.” These polymers typically have a core and branching to a spherical, hemispherical or other 3-dimensional shape, which provides a surface which is functionalized. Reactive sites on this functionalized surface are then blocked and/or further functionalized to provide sites for attachment of starting materials, which are then reacted, characterized and isolated.

These polymers have a molecular diameter of about 100 to about 10,000 Å, which allows for isolation by ultra filtration and washing. The size of the polymers also allows for them to be soluble or suspended, thus solution based organic chemistry apparatus can be used, with simplified procedures of support based synthesis. As a result, the method of the invention provides for recovery of unreacted starting materials and characterization in situ, as well as combinatorial synthesis of compounds (producing large numbers of molecular variants in one reaction, or in several related reactions). Thus the method provides vast increases in productivity for the skilled artisan in organic chemistry.

For example the invention contemplates NMR, IR, UV or other spectra of the molecules made, while still on the “support.” In addition, homogenous or continuous phase screening can also occur without purification of the molecule per se.

### THREE-DIMENSIONAL POLYMERS

As used herein the term “Three-dimensional polymer” refers to a polymer that has

1) a central core;

2) reactive sites on its surface (and hence is considered by the art as a “living polymer”); and

3) a three-dimensional shape.

The shape need not be spherical, and the polymer need not be in suspension or solution. For example, it may be attached to another solid, such as a metal, glass or polymer solid, such as a reaction vessel. In addition, the shape of the polymer may be altered by the core unit. For example, replacing an amine core unit with a substituted amine (NH₂R) generates a “dumbbell shaped” polymer. Finally, there can be inter-polymer linkages which generate bridged polymers and polymer clusters.

The term “combinatorial synthesis” is recognized in the art and refers to the method of making molecules where from one starting material, a host of others are made in a parallel fashion.

Underivatized three-dimensional polymers-making

The polymers used in this invention are known in the art, or are made by known methods. The reactive sites appearing on the surface of the polymer result from the judicious choice of starting material used.

Examples of methods for making these three-dimensional polymers are disclosed in the art. For example, there are two basic approaches to synthesize a polymer, a divergent method and a convergent method.

Divergent Synthesis

Divergent method involves constructing branch cells around an initial core. For example, in the synthesis of “Three-dimensional” polyamidoamine (PAMAM) polymers (Tomalia D. A., Aldrichimica Acta, Vol. 26, No. 4, 1993, p 91), involves the reaction of ammonia with methyl acrylate to produce a triester intermediate. The addition of the triester to a large excess of ethylenediamine produces a terminal triamine core cell. Repeating these steps leads to a hexaamine, a “generation one” polymer. Continuing this sequence produces increasingly higher generations.

Other polymers produced by this approach include:

poly(ethers) (Hall H., Padias A., McConnel R., Tomalia D. A., J. Org. Chem. 1987, 52, 5305), preferably poly(arylalkyl ethers), poly(ary/lazacrown ethers);

poly(siloxanes) (Uchida H., Kabe Y., Yoshino, K., Kawamata A., Tsumuraya T. Masamune S., J. Am. Chem. Soc. 1990, 112, 7077).

poly(thioethers) (Tomalia D. A. Padia A. Hall H. K. Jr. Polym. Prepr., Am. Chem. Soc. Div. Polym. Chem. 1989, 30, 119),

poly(amidoalcohol),

poly(amines), preferably poly(ethylene amines)

poly(phosphonium),

polyesters, preferably poly(arylester),

polyalkenes and polyarenes, preferably poly(arylene),

poly(alkanes), and

poly(nucleic acids).

Convergent Method

The convergent synthesis begins with monomers that will ultimately appear on the surface of the polymer and adds monomers “inwardly.” It is a convergent method because it proceeds to make several “reagents which are actually parts of the larger molecule, that are ultimately attached to the “core” or central monomer.

Typically, one starts with a monomer which has surface functional groups which are protected so that they do not react in the making of the polymer, and a reactive functional group, which will ultimately be buried in the polymer. The monomer is then coupled to another of the same or different monomer. This reaction provides an oligomeric “reagent” where at least two monomers have reacted with another, or perhaps different monomer.

The “reagent,” with protected surface functional groups (or groups that will not participate in side reactions, such as in the next reaction in preparing the polymer), and a protected functional group is a “first level intermediate.” The protected functional group is then deprotected, forming a reactive moiety. The “reagent” (i.e., deprotected “intermediate”) is then reacted with a monomer, which can be the same or different to generate a “second level intermediate,” which can then be deprotected and reacted with another monomer (same or different). The number of generations will alter the size of the polymer. This process is repeated until an intermediate with desired number of “levels.” This ultimate intermediates preferably have a single reactive functional group, which is then coupled to a monomeric reagent with multiple functional groups (which serves as an “anchoring core”), producing the polymers useful for the invention.

For example, polymers produced by this approach include:

Poly(haloalkylaryl ether) (Percec V., Kawasumi M., Macromolecules 1992, 25 3843);

Poly(arylester) (Kwock E. W., Neenan T. X., Miller T. M., J. Chem. Soc., 1991, 113, 4252);

Poly(arylene) (Miller T. M., Neenan T. X., Zayas R., Bair H. E., J. Am. Chem. Soc., 1992,114, 1018); and

Poly(arylacetylenic) (Moore J. S., Xu Z., Macromolecules, 1991, 24, 5893).

Of course, the skilled artisan envisions that mixtures of the polymers listed above are easily made given the guidance of the specification and the knowledge readily available in the art. Variation in the polymer building blocks, branch cell multiplicity, and the number of generations will allow the design of specific polymers suitable for various reactions and reaction conditions.

As used herein the term “number of generations” refers to the number of repeating steps in the synthesis of the polymer. Since the number of generations is related to the number of “layers of monomer” added to the polymer, the number of generations also describes the size and mass of the polymer, given the monomer structure.

As used herein the term “branch cell multiplicity” refers to the number of reactive sites in the branch cell repeating unit. The branch cell multiplicity directly affects the number of terminal groups, the number of repeating units, and the molar mass of the polymer as a function of generation.

Functionalization of the three-dimensional polymer

A “blocking moiety” as used herein, is a moiety that is covalently linked to the polymer that does not provide an active site for reactions to occur. For example, where the living polymer has a moiety that will react with an amine, preferably the blocking group will have at one end an amine, and no other reactive groups. For steric reasons the blocking moiety may have more than one reactive site if all of the reactive sites on the blocking moiety will react with the living polymer and only unreactive sites will be exposed to the surface of the 3-dimensional polymer.

A “reactive moiety” as used herein, refers to a moiety that is reacted with the surface of the living polymer, preferably the living polymer with most of the reactive sites blocked to control derivatization. It will have an end which bonds to the surface of the polymer, and a second end having one or more reactive groups attached to it which will serve as an “anchor” for the compound to be made. Thus it is bifunctional.

Preferred reactive groups for attaching small molecules to the polymer include —CH₂Br, CH₂Cl, —NH₂, —NHR, —OH, —CHO, —COOH, —SH, or others known in the art. The functional groups on the surface can also be easily modified, using standard chemical techniques.

The loading of reactive sites is controlled by changing the ratio of inert blocking groups to the functional groups. In addition, variation in reactive sites are obtained by changing the ratio of “blocking moieties” and reactive moieties.

The starting materials used in preparing the invention are known, made by known methods, or are commercially available as a starting materials.

The polymers may then be derivatized by adding inert blocking groups or protecting groups. These may be found in the literature and will be apparent to the skilled artisan.

It will be apparent to the skilled artisan that these reactions can be supplemented and modified using reaction chemistry found in or modified from the literature. Furthermore, other known methods and starting materials from the literature can be employed in making the compounds of the invention. Thus the list of schemes above is illustrative, but not exhaustive. They are meant to provide the skilled artisan with guidance as to how the compounds can be made. Since other methods can be used to make them, and these methods are within the purview of the skilled artisan, the methods shown do not limit the claims in any way, nor are they intended to limit the claims.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg Advanced Organic Chemistry (2 vol.) and Trost and Fleming Comprehensive Organic Synthesis (6 vol.).

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations are found, for example, in T. Greene Protecting Groups in Organic Synthesis.

The reaction products of each reaction step are characterized by routine analytical techniques such as H-1, C-13 NMR spectroscopy, mass spectrometry, IR spectroscopy and the like. This is possible since the products of the reaction (including the three-dimensional polymer itself) are suspendable or soluble. The analytical techniques described above and applied to purified organic molecules are discussed in standard text books (e.g., Introduction to Organic Chemistry by Streitwieser). In this invention, the same techniques can be applied to the polymer/reaction product complex, without purification. For example, the success of a reaction step adding aromatic functionality to a small molecule can be confirmed by the observation of additional C-13 NMR signals in the aromatic region.

Use of the three-dimensional polymer with product in screening

The solubility or suspendibility of the polymers allows biological screening without purification of the reaction products. (Of course, the polymer does not preclude such purification either.) The assay procedures can include;

(1) those that rely on affinity purification with an immobilized target receptor,

(2) those in which a soluble receptor binds to tethered ligands, and

(3) those in which soluble compounds are tested for activities, either directly or in competition assays.

### EXAMPLES

The following non-limiting examples provide details for the preparation of the derivatized three-dimensional polymer and their use in organic synthesis. Since these examples are illustrative, it is contemplated that the skilled artisan can prepare variations of these examples within the scope of the claims. Thus these examples provide the skilled artisan with illustrative, rather than exhaustive methodologies to carry out invention and its method.

### Example 1

Preparation of the combinatorial support

A polymer is made using the method of Tomalia above with the following parameters: a core structure of N—[(CH₂)₂C(O)]₃—, a repeating unit structure of —NHCH₂CH₂N[CH₂CH₂C(O)]₂—, a molecular weight of 28600 (6 generations), and 96 —CH₂CH₂COOCH₃ functional groups on the surface of each polymer. The surface of the polymer is then modified by reacting 50 g polymer with a mixture of 16.5 g NH₂CH₂CH₂CH₃ and 4 g NH₂CH₂CH₂OH in methanol at 45° C. to reduce the number of the reactive sites. NMR and mass spectroscopy are used to monitor the reaction progress. Excess reagents and solvent are then removed under high vacuum. The resulting polymer has a molecular weight of 34600 and 20 —CH₂CH₂OH functional groups on each polymer molecule surface. The loading of reactive sites is 580μ equivalents/g.

### Example 2

Combinatorial synthesis

The following combinatorial synthesis is carried out using the polymer of Example 1 with —CH₂CH₂OH reactive sites.

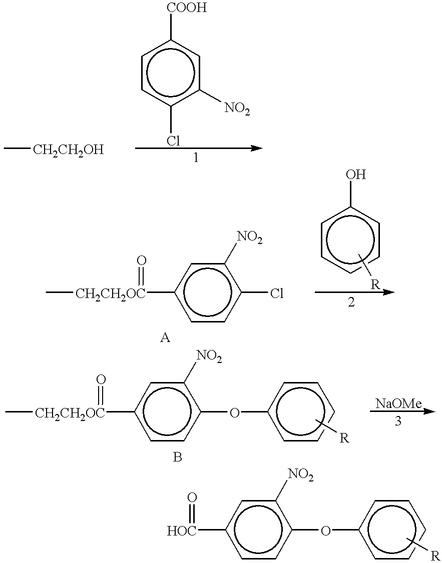

The reactions are carried out under homogeneous solution conditions with the easy separation and purification offered by polymer supported combinatorial chemistry.

### Example 3

Characterization of the reaction products

The success of step one is confirmed by the presence of additional C-13 NMR signals at aromatic region with corresponding intensities. In the mass spectrum, an addition peak with a mass of polymer+183 m/e verifies the success of the reaction step in Example 2, using the unreacted three-dimensional polymer in 2A to determine the mass of the support, and the reactant.

### Example 4

Recovery of product and starting materials

The product can also be easily separated by cleaving small molecules from the polymer supports. The polymer, therefore, is recovered. The reaction step 3 in example 2 describes cleaving phenol molecules from polymers, such as those in Example 1. The phenol molecules are separated by ultra-filtration using AMICON SR3 concentrator. The polymer, with

—CH₂CH₂OH as reactive sites, is then re-suspended and washed with methanol for future use.

### Example 5

Variation in the loading of the reactive sites

The loading of the reactive sites is easily controlled to suit a particular combinatorial synthesis. A polymer was prepared according to the same procedure as described in example 1, the only difference being that the amounts of NH₂CH₂CH₂CH₃ and NH₂CH₂CH₂OH in this case are and 19.5 g and 1.0 g, respectively. The resulting polymer has a molecular weight of 34600 and 5 —CH₂CH₂OH functional groups on the surface of each polymer. The loading of reactive sites is 145μ equivalents/g.

### Example 6

Variation in polymer size

The polymer size can also be varied. A generation five polymer of Example 1 has a molecular weight of 14100 and 48 —CH₂CHCOOCH₃ functional groups on the surface of each polymer. The reaction of 50 g of this polymer with 16.9 g NH₂CH₂CH₂NH₂ and 4.2 g NH₂CH₂CH₂OH under the conditions described in Example 1 produces in a polymer with a molecular weight of 17000. The loading of reactive sites is 560μ equivalents/g.

### Example 7

Modification of surface functional groups

The surface functional groups is easily modified to suit various combinatorial synthesis. The —CH₂CH₂OH on the polymer surface is oxidized to —CH₂CH₂CHO by reacting 50 g of the polymer of Example 1 with 69 g pyridinium dichromate in 500 ml dichloromethane at room temperature. The excess reagents are removed by ultra-filtration as described in Example 4. This same procedure using dimethylformamide as solvent instead of dichloromethane converts —CH₂CH₂OH into —CH₂CH₂COOH. The conversion of the —CH₂CH₂OH functional groups into —CH₂CH₂Br is accomplished by refluxing 50 g polymer in 200 ml 47% HBr aqueous solution for 2.5 hrs. After the reaction mixture is cooled, the excessive HBr is removed by ultra-filtration.

### Example 8

Variation in Polymer shape

The shape of polymers is influenced by the core unit. In example 1, when NH₂CH₂CH₃ is used as the core unit instead of NH₃, the resultant polymer has a “dumbbell” shape. The reaction of 50 g (0.17 eqls.) polymer with CH₂CH₂COOCH₃ surface functional groups as described in Example 1 with 9 g NH₂CH₂CH₂NH₂ introduces cross linking or bridging between polymers resulting in bridged polymers or polymer clusters.

### Example 9

Use of Materials in Biological Screening

The solubility of polymers also allows for biological screening of the materials without purification as well. Assay procedures include (1) those that rely on affinity purification with an immobilized target receptor, (2) those in which a soluble receptor binds to tethered ligands, and (3) those in which soluble compounds are tested for activities, either directly or in competition assays.

Modification of the preceding embodiments is within the scope of the skilled artisan in formulation, given the guidance of the specification in light of the state of the art.

All references described herein are hereby incorporated by reference.

While particular embodiments of this invention have been described, it will be obvious to those skilled in the art that various changes and modifications of this invention can be made without departing from the spirit and scope of the invention. It is intended to cover, in the appended claims, all such modifications that are within the scope of this invention.

What is claimed is:

1. A method of making a support for a chemical reaction characterized in that it comprises:
a. preparing a suspension or solution of polymer with a diameter of 100 to 10,000 Å,
b. derivatizing the polymer with blocking and reactive moieties thus providing a soluble or suspendable support for a reaction.

2. A method of making one or more molecules using the support of claim 1, comprising:
a. covalently attaching a starting material to the reactive moiety,
b. reacting that starting material with one or more reactants.

3. A reaction mixture comprising the material of claim 2.

4. A method of monitoring the progress of a reaction comprising exposing a suspension of the reaction mixture of claim 3 to a solution phase analytical method.

5. The method of claim 4 wherein the method is spectroscopy.

6. The method of claim 4 wherein the method is chromatography.

7. A method of purifying the molecule made using the method of claim 2 comprising one or more cycles of washing and ultrafiltration of the reaction mixture.

8. A method of screening the molecule according to claim 2 comprising:
a. exposing the molecule to a biological material; and
b. determining the response.
